(19) United States
(12) Patent Application Publication
(10) Pub. No.: US 2022/0218860 A1
(43) Pub. Date: Jul. 14, 2022

(54) INDANE DERIVATIVES FOR MALODOR COUNTERACTION

(71) Applicant: FIRMENICH SA, Satigny (CH)

(72) Inventors: Matthew Rogers, Briarcliff Manor, NJ (US); Christian Margot, Satigny (CH); Christine Vuilleumier, Satigny (CH); Ben Smith, Satigny (CH); Sanja Fitzgerald, Satigny (CH); Maud Reiter, Satigny (CH); Stefano Nicolai, Satigny (CH)

(21) Appl. No.: 17/591,250

(22) Filed: Feb. 2, 2022

Related U.S. Application Data
(63) Continuation of application No. 15/736,857, filed on Dec. 15, 2017, now abandoned.

(30) Foreign Application Priority Data
Jul. 7, 2015 (EP) 15175717.6

(51) Int. Cl. A61L 9/01; C11B 9/00; C11D 3/00; C11D 3/20; A61L 9/012; C07C 43/174; C07C 35/32; C07C 43/168

(52) U.S. Cl. CPC A61L 9/01; C11B 9/0049; C11D 3/0068; C11D 3/2068; C07C 2602/08; C07C 43/1747; C07C 35/32; C07C 43/168; C11D 3/2034; A61L 9/012

(57) ABSTRACT

The present invention relates to the field of malodor counteraction. More particularly, it concerns malodor masking ingredient having an indane moiety (as defined in formula (I)), as well as malodor masking compositions comprising such ingredients.

### TECHNICAL FIELD

The present invention relates to the field of perfumery. More particularly, it concerns malodor masking compositions and/or ingredients, as well as methods to counteract or mask malodors and perfuming compositions having odor masking properties.

### PRIOR ART

Smells perceived as malodorous exist in many environments and are experienced in our daily life. The odorants eliciting this bad perception are created in any environment. In particular one may cite commercial and residential environment malodors which can for example be generated by waste products, trash receptacles, toilets, cat litter, and food handling and processing. Toilet (in particular feces), kitchen and body malodor, are just a few of the common environmental sources of malodors in daily life. Said malodors are usually complex mixtures of more than one malodorant compound which may typically include various amines, thiols, sulfides, short chain aliphatic and unsaturated acids, e.g. fatty acids, and their derivatives.

Residential or body related malodors are typically due to various chemical compounds such as indole, skatole, and methanethiol found in feces malodor; piperidine and morpholine found in urine; pyridine and triethyl amine found in kitchen and garbage malodors; and short chain fatty acids, such as 3-methyl-3-hydroxyhexanoic acid, 3-methylhexanoic acid or 3-20 methyl-2-hexenoic acid, found in axillary malodors.

Obviously such malodors are not pleasant for humans and therefore there is a constant need for malodor counteracting technologies (MOC) decreasing or suppressing the perception of malodors. Various approaches exist to achieve such a goal with MOC compositions, and include i) odor coverage (which relates to superimposing the malodor with a pleasant stronger odor) and/or ii) odor antagonism (which relates to either suppressing or decreasing the perception by blocking the olfactory receptors involved in decoding the odorants perceived as bad or iii) odor sequestration produced by the chemical or physical interception of the malodorant molecules or by preventing their formation).

However the task is generally very difficult because the chemicals responsible for the malodor elicit extremely powerful smells and can have much lower detection thresholds than the odorants used to mask them. Therefore one has to use excessive amounts of MOC composition/compounds to achieve an acceptable malodor counteracting action.

The prior art reports some MOC compositions, and in particular one can mention EP 1393752, which reports about the use of some phenyl derivatives as malodor counteracting ingredient against kitchen, trash bin or urine malodor.

The aim of the present invention is to provide a MOC composition capable of being highly effective against the malodors of feces.

The present invention's compounds have never been cited as useful MOC ingredients, and only a few of them have been quoted in the literature; in particular one may cite EP 1022265 which reports the standard use as perfuming ingredient for some of the present derivatives; as mentioned above, a perfuming use is different from a MOC use.

### DESCRIPTION OF THE INVENTION

We have now surprisingly discovered that a compound of formula

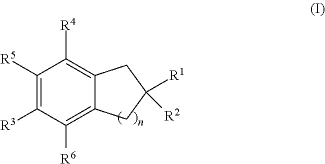

wherein n represents 1 or 2;
R¹ represents a hydrogen atom or a methyl or ethyl group;
R² represents a CH₂OR⁷ or a R⁸CO group, R⁷ being a hydrogen atom or a C₁₋₃ hydrocarbon group or a R⁸CO group, and R⁸ being a hydrogen atom or a C₁₋₃ hydrocarbon group;
R³ represents a hydrogen atom or a C₁₋₄ hydrocarbon group or a C₁₋₂ alkoxyl group; and
each of R⁴, R⁵ and R⁶ represents, independently from each other, a hydrogen atom or a C₁₋₃ alkyl group;
can be used for counteracting toilet and in particular fecal type malodors.

According to any embodiment of the invention, said invention's compound can be one wherein n represents 1 or 2;
R¹ represents a hydrogen atom or a methyl or ethyl group;
R² represents a CH₂OR⁷ or a R⁸CO group, R⁷ being a hydrogen atom or a C₁₋₃ alkyl group or a R⁸CO group, and R⁸ being a hydrogen atom or a C₁₋₃ alkyl group;
R³ represents a hydrogen atom or a C₁₋₄ hydrocarbon group or a C₁₋₃ alkoxyl group; and
each of R⁴, R⁵ and R⁶ represents, independently from each other, a hydrogen atom or a methyl group.

According to any embodiment of the invention, said invention's compound can be of formula

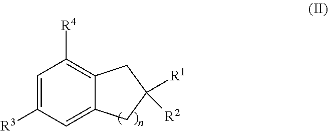

wherein n represents 1 or 2;
R¹ represents a hydrogen atom or a methyl group;
R² represents a CH₂OR⁷ or a R⁸CO group, R⁷ being a hydrogen atom or a methyl or ethyl group or a R⁸CO group, and R⁸ being a methyl or ethyl group;
R³ represents a hydrogen atom or a C₁₋₄ alkyl group; and
R⁴ represents a hydrogen atom or a methyl group.

According to any embodiment of the invention, said invention's compound can be of formula

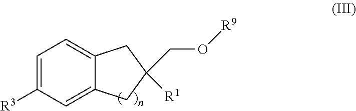

wherein n represents 1 or 2;
R¹ represents a hydrogen atom or a methyl group;
R⁹ represents a hydrogen atom or a methyl or ethyl group or a CH₃CO group; and
R³ represents a hydrogen atom or a methyl or ethyl group.

According to any embodiment of the invention, said invention's compound can be of formula

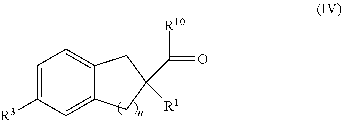

wherein n represents 1 or 2;
R¹ represents a hydrogen atom or a methyl group;
R¹⁰ represents a hydrogen atom or a methyl or ethyl group; and
R³ represents a hydrogen atom or a methyl or ethyl group.

According to any embodiment of the invention, said invention's compound can be a compound wherein n is 1.

According to any embodiment of the invention, said invention's compound can be a C₁₋₃ compound.

For the sake of clarity, the compound (I) can be used in the form of a racemate, i.e. having an e.e. (enantiomeric excess) equal to 0, or as an enantiomerically enriched form, i.e. having an e.e. above 0, preferably above 50, or even above 80 or 95. According to any embodiment of the invention, said invention's compound can be used in the form of a racemate.

According to any embodiment of the invention, said invention's compound can be either characterized by a pleasant odor (according to the standard of the perfumery art, well known by a person skilled in the art), e.g. being known as being a perfuming ingredient, or by having a weak or undetectable odor. For the sake of clarity, by “weak or undetectable compound” we mean a compound that has either no odor or an odor perception threshold well above its vapor pressure.

As specific, but non-limiting, examples of the invention's compounds, one may list the following chemicals in Table 1:

TABLE 1
Specific examples of invention's compounds
Compound structure and name

2,5-dimethyl-2-indanemethanol (compound 1)
Odor: established perfuming ingredient
(−)-(R)-2,5-dimethyl-2-indanemethanol (compound 1R)
Odor: established perfuming ingredient
(−)-(S)-2,5-dimethyl-2-indanemethanol (compound 1S)
Odor: established perfuming ingredient
(2,5-dimethyl-2,3-dihydro-1H-inden-2-yl)methyl methyl ether (compound 2)
Odor: medicinal, floral
(2-methyl-2,3-dihydro-1H-inden-2-yl)methanol (compound 3)
Odor: weak, lily of the valley
(5-methyl-2,3-dihydro-1H-inden-2-yl)methanol (compound 4)
Odor: floral
(2-methyl-2,3-dihydro-1H-inden-2-yl)methyl acetate (compound 5)
Odor: Slightly metallic, waxy
1-(2,5-dimethyl-2,3-dihydro-1H-inden-2-yl)ethanone (compound 6)
Odor: fatty
(2,6-dimethyl-1,2,3,4-tetrahydro-2-naphthalenyl)methanol (compound 7)
Odor: very weak or odorless
5-ethyl-2-methyl-2-indanmethanol (compound 8)
Odor: very weak or odorless
(2,4,5-trimethyl-2,3-dihydro-1H-inden-2-yl)methanol (compound 9)
Odor: weak, lily of the valley
(2,5-dimethyl-2-indanyl)methyl acetate (compound 10)
Odor: floral
2-ethyl-5-methyl-2-indanmethanol (compound 11)
Odor: very weak or odorless
5-isopropyl-2-methyl-2-indanmethanol (compound 12)
Odor: weak, linalool
5-tert-butyl-2-methyl-2-indanmethanol (compound 13)
Odor: weak, floral
2,5,6-trimethyl-2-indanmethanol (compound 14)
Odor: lily of the valley
2,4-dimethyl-2-indanmethanol (compound 15)
Odor: weak, lily of the valley
(2,4,6-trimethyl-2,3-dihydro-1H-inden-2-yl)methanol (compound 16)
Odor: weak, lily of the valley
(5-methoxy-2-methyl-2,3-dihydro-1H-inden-2-yl)methanol (compound 17)
Odor: weak, lily of the valley

According to any embodiment of the invention, said invention's compound is a C₁-C₁₃ compound.

According to a particular embodiment of the invention, the compounds of formula (I) are 2,5-dimethyl-2-indanemethanol, (2,5-dimethyl-2,3-dihydro-1h-inden-2-yl)methyl methyl ether, (2-methyl-2,3-dihydro-1h-inden-2-yl)methanol, (5-methyl-2,3-dihydro-1h-inden-2-yl)methanol, (2-methyl-2,3-dihydro-1h-inden-2-yl)methyl acetate, 1-(2,5-dimethyl-2,3-dihydro-1h-inden-2-yl)ethanone, (2,4,6-trimethyl-2,3-dihydro-1H-inden-2-yl)methanol and/or (2,6-dimethyl-1,2,3,4-tetrahydro-2-naphthalenyl)methanol.

According to a particular embodiment of the invention, the compounds of formula (I) are 2,5-dimethyl-2-indanemethanol, (5-methyl-2,3-dihydro-1h-inden-2-yl)methanol, (2,4,6-trimethyl-2,3-dihydro-1H-inden-2-yl)methanol, (2-methyl-2,3-dihydro-1h-inden-2-yl)methyl acetate and/or 1-(2,5-dimethyl-2,3-dihydro-1h-inden-2-yl)ethanone, and in particular 2,5-dimethyl-2-indanemethanol, (5-methyl-2,3-dihydro-1h-inden-2-yl)methanol and/or 1-(2,5-dimethyl-2,3-dihydro-1h-inden-2-yl)ethanone.

The compounds of formula

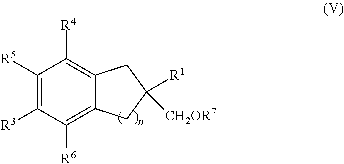

wherein n represents 1 or 2;
R¹ represents a hydrogen atom or a methyl or ethyl group;
R⁷ being a C₁₋₃ hydrocarbon group;
R³ represents a hydrogen atom or a C₁₋₄ hydrocarbon group or a C₁₋₃ alkoxyl group; and
each of R⁴, R⁵ and R⁶ represents, independently from each other, a hydrogen atom or a C₁₋₃ alkyl group;

provided that 2-methoxy-2,3-dihydro-1H-indene is excluded;

are also novel compounds and therefore an object of the present invention. It is understood that for formula (V) the above-mentioned meaning of the various R groups, or n, does apply too.

As mentioned above, the invention concerns the use of the above-defined compounds as MOC ingredients, e.g. to modify, suppress, reduce, decrease or mask the sensory perception of toilet, and in particular feces, malodors. In other words, it concerns a method to modify, suppress, reduce, decrease or mask a toilet, and in particular feces, malodor, which method comprises the step of releasing into the air or over a surface, or to the malodor source, an effective amount of at least an invention's compound. By “use of an invention's compound” it has to be understood here also the use of any MOC composition containing a compound (I) and which can be advantageously employed.

As non-limiting examples of feces malodor one may cite any malodor present in a toilet room or the similar, including, but not limited to: odors present immediately after the use of the toilet; lingering toilet odors; and, moldy or musty odors that often originate in damp areas of the bathroom such as around the toilet.

According to any embodiment of the invention, said toilet, and in particular feces, malodor can be described by adjectives such as dung, fecal, tar and/or animal odor type.

According to any embodiment of the invention, the invention's compound is used, as described above, and in particular against said malodors which are generated by the presence of skatole, C₁₋₇ aliphatic carboxylic acids, methyl morpholines, thioglycolic acid, cresols, C₁₋₄ dialkyl sulfide or disulfide or trisulfide, indole, and/or C₁₋₇ thiols or mixtures thereof. In particular generated by the presence of skatole, p-cresol, dimethyl sulfide or disulfide or trisulfide, indole, or mixtures thereof.

According to any embodiment of the invention, the releasing mentioned above can be obtained through the application of any known consumer product relevant for the targeted surface.

According to any embodiment of the invention, said surface is a bathroom, a toilet, a trash (e.g. for napkins).

Accordingly, the present invention refers in a further embodiment to the non-therapeutic use of an invention' s compound for the reduction of the sensory perception of malodor by a human.

Without being bond by theory, it is believed, that the invention's compound, as hereinabove defined, do act through a mechanism related to odor antagonism (e.g. through blocking olfactory receptors) and optionally odor coverage. We find support for this hypothesis by the surprising finding that most known lily-of-the-valley or muguet type odorants, a class of odorant eliciting a perception closely related to many of compounds described in the present invention, fail to achieve a similar reduction of the negative odor character. Odorants such as 3-(4-tert-butylphenyl)-2-methylpropanal, 3-(4-tert-butylphenyl)propanal or 3-(3,3-dimethyl-2,3-dihydro-1h-inden-5-yl)propanal, with a typical lily-of-the-valley or muguet smell similar to many of the indane derivatives described here, fail to produce such malodor reduction. In binary mixtures, the fecal and animal note remains clearly perceptible.

Said invention's compound, which in fact can be advantageously employed as MOC compound, is also an object of the present invention.

It is understood by a person skilled in the art that the invention's compound, as defined herein, may be added into an invention's composition in neat form, or in a solvent, or they may first be modified, for example by entrapped with an entrapment material such as for example polymers, capsules, microcapsules, nanocapsules, liposomes, precursors, film formers, absorbents such as for example by using carbon or zeolites, cyclic oligosaccharides and mixtures thereof, or they may be chemically bound to substrates which are adapted to release the compounds upon application of an exogenous stimulus such as light, enzymes, or the like. Therefore when referring to the invention's compound it is also intended any of its form mentioned above.

Therefore, another object of the present invention is a MOC composition comprising:
i) as a MOC ingredient, at least one invention's compound as defined above;
ii) at least one ingredient selected from the group consisting of a perfumery carrier and a perfumery base; and
iii) optionally at least another MOC compound; and
iv) optionally at least one perfumery adjuvant.

It is understood that said MOC composition, by its nature, could be also a perfuming one.

By “perfumery carrier” we mean here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company).

As solid carriers one may cite, as non-limiting examples, absorbing gums or polymers, or yet encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs- and Geliermittel in Lebensmitteln, Band 2 der Schriftenreihe Lebensmittelchemie, Lebensmittelqualität, Behr's Verlag GmbH & Co., Hamburg, 1996. The encapsulation is a well-known process to a person skilled in the art, and may be performed, for instance, using techniques such as spray-drying, agglomeration or yet extrusion; or consists of a coating encapsulation, including coacervation and complex coacervation technique.

By “perfumery base” we mean here a composition comprising at least one perfuming co-ingredient.

Said perfuming co-ingredient is not of formula (I). Moreover, by “perfuming co-ingredient” it is meant here a compound, which is used in a perfuming preparation or a composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of his general knowledge and according to intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

By “other MOC compounds” we mean here a material which is already known for a MOC activity and is commonly used in the industry for such use. Said other MOC compound can be included to further boost, or complement, the MOC activity of the invention's MOC composition. Said other MOC compound can be effective through any mechanism (e.g. odor coverage, antagonism or sequestration).

Said other MOC compounds include, but are not limited to, antimicrobial agents, malodor absorbers, chemical neutralisers e.g. acid-base reagents, thiol traps, etc, odor blockers, cross-adaptation agents e.g. as disclosed in U.S. Pat. No. 5,538,719 incorporated herein by reference, malodor complexation agents e.g. various cyclodextrins.

Examples of antimicrobial agents include, but are not limited to, metal salts such as zinc citrate, zinc oxide, zinc pyrethiones, and octopirox; organic acids, such as sorbic acid, benzoic acid, and their salts; parabens, such as methyl paraben, propyl paraben, butyl paraben, ethyl paraben, isopropyl paraben, isobutyl paraben, benzyl paraben, and their salts; alcohols, such as benzyl alcohol, phenyl ethyl alcohol; boric acid; 2,4,4′-trichloro-2-hydroxy-diphenyl ether; phenolic compounds, such as phenol, 2-methyl phenol, 4-ethyl phenol; essential oils such as rosemary, thyme, lavender, eugenol, geranium, tea tree, clove, lemon grass, peppermint, or their active components such as anethole, thymol, eucalyptol, farnesol, menthol, limonene, methyl salicylate, salicylic acid, terpineol, nerolidol, geraniol, and mixtures thereof.

Examples of malodour absorbers include, but are not limited to molecular sieves, such as zeolites, silicas, aluminosilcates, and cyclodextrins; and organic absorbents, such as for example, activated charcoal, dried citrus pulp, cherry pit extract, corncob, and mixtures thereof.

An invention's composition consisting of at least one an invention's compound and at least one perfumery carrier and at least another MOC ingredient represents a particular embodiment of the invention.

It is useful to mention here that the possibility to have, in the compositions mentioned above, more than one compound of formula (I) is important as it enables the person skilled in the art to prepare MOC compositions possessing an activity fine-tuned toward the targeted malodor or source of malodor, creating thus new tools for his work.

For the sake of clarity, it is also understood that any mixture resulting directly from a chemical synthesis, e.g. a reaction medium without an adequate purification, in which the compound of the invention would be involved as a starting, intermediate or end-product could not be considered as a MOC composition according to the invention as far as said mixture does not provide the inventive compound in a suitable form. Thus, unpurified reaction mixtures are generally excluded from the present invention unless otherwise specified.

Furthermore, the invention's compound can also be advantageously used in any consumer product for which is may be useful to have an MOC activity at least. Consequently, another object of the present invention is represented by a MOC consumer product comprising, as an active ingredient, at least one invention's compound or composition, as defined above.

The invention's compound or composition can be added as such or as part of an invention's a MOC composition.

It is understood that said MOC consumer product, by its nature can also be a perfuming one.

For the sake of clarity, it has to be mentioned that, by “MOC, and optionally perfuming, consumer product” or the similar, it is meant a consumer product which is expected to deliver at least a MOC effect, and optionally also a pleasant perfuming effect, to the surface to which it is applied (e.g. skin, hair, textile, or home surface, but also air). In other words, a consumer product according to the invention is a perfumed consumer product which comprises the functional formulation, as well as optionally additional benefit agents, corresponding to the desired consumer product, e.g. a detergent or an air freshener, and an effective amount of at least one invention's compound or composition. For the sake of clarity, said consumer product is a non-edible product.

The nature and type of the constituents of the MOC consumer product do not warrant a more detailed description here, which in any case would not be exhaustive, the skilled person being able to select them on the basis of his general knowledge and according to the nature and the desired effect of said product.

Non-limiting examples of suitable perfuming consumer product can be:
a fabric care product, such as a liquid detergent, a powder detergent, detergent tablets, a detergent bar, a detergent paste, a liquid fabric softener, fabric softener sheets, a fabric scent booster, a laundry pre-treatment, a fabric refresher, an ironing water, a laundry bleach, a carpet powder or a carpet cleaner; the uses for this type of product would be particularly beneficial in the cases where standard water available to the consumers could be associated with malodor as described (e.g. putrid waters);
a toilet paper or napkin;
an air freshening product, such as an air freshener spray, a gel air freshener, a liquid-wick air freshener, a solid air freshener comprising a porous substrate (such as a paper or card blotter, a porous ceramic, or a porous plastic), a liquid or gel air freshener comprising a permeable membrane, an electrically operated air freshener, and a dual purpose air freshener/disinfectant spray; and/or
a surface care product, such as an all-purpose cleaner, a furniture polish, a wood floor cleaner, a toilet care product (such as a toilet bowl cleaning liquid, an in-cistern toilet cleaner, a toilet rim block, or a toilet rim liquid); a pet-litter.

Some of the above-mentioned MOC consumer products may represent an aggressive medium for the invention's compound, so that it may be necessary to protect the latter from premature decomposition, for example by encapsulation or by chemically bounding it to another chemical which is suitable to release the invention's ingredient upon a suitable external stimulus, such as an enzyme, light, heat or a change of pH.

It should be appreciated by those skilled in the art that the conception and the specific embodiments disclosed might be readily utilized as a basis for modifying or formulating other formulations for carrying the same purposes of the present invention. It should also be realized by those skilled in the art that such equivalent formulations do not depart from the spirit and scope of the invention as set forth in the appended claims.

The proportions in which the compound according to the invention can be incorporated into the various aforementioned products or compositions vary within a wide range of values. These values are dependent on the nature of MOC consume product and on the desired organoleptic effect as well as the nature of the co-ingredients in a given composition when the compounds according to the invention are mixed with other ingredients, solvents or additives commonly used in the art.

For example, in the case of perfuming compositions, typical concentrations are in the order of 0.01% to 20%, or even 1% to 10%, by weight, or even more, of the compound of the invention based on the weight of the composition into which they are incorporated. Concentrations lower than these, such as in the order of 0.01% to 2% by weight, can be used when these compounds are incorporated into MOC consumer products, percentage being relative to the weight of the consumer product.

In particular, the concentration of MOC compound according to the invention used in the various aforementioned consumer products varies within a various wide range of values depending on the nature of the consumer product.

### DESCRIPTION OF THE DRAWINGS

FIG. 1: Ca²⁺ imaging traces of individual indole-responsive olfactory sensory neurons and their inhibition to Compound 1, Compound 2 or Compound 3 (MOC) are shown in FIG. 1 plots (A, C, E). Inhibition of the population of indole-responsive olfactory sensory neurons is shown in FIG. 1 plots (B, D, F).

Open circles are individual olfactory neuron modulation values that lie outside 95^{th} percentile.

FIG. 2: Radar graph reporting the duplicated assessment of 3 odor descriptors (Animal/Fecal/Tar, Pleasantness and Freshness) for indole alone ( - - - :Indole alone) and indole combined to Compound 1 (- - - : indole alone+Compound D.

The invention's compounds can be prepared according to a method known in the literature, and the compounds of formula (V) can be obtained by a standard alkylation of the corresponding alcohol.

### EXAMPLES

The invention will now be described in further detail by way of the following examples, wherein the abbreviations have the usual meaning in the art, the temperatures are indicated in degrees centigrade (° C.); the NMR spectral data were recorded in CDCl₃ (if not stated otherwise) with a 360 or 400 MHz machine for ¹H and ¹³C, the chemical shifts δ are indicated in ppm with respect to TMS as standard, the coupling constants J are expressed in Hz.
2,5-Dimethyl-2-indanemethanol (compound 1); 2-Methyl-2-indanemethanol (compound 3); 5-methyl-2-indanemethanol (compound 4); (2-Methyl-2,3-dihydro-1H-inden-2-yl)methyl acetate (compound 5); 5-ethyl-2-methyl-2-indanmethanol (compound 8); 5-isopropyl-2-methyl-2-indanmethanol (compound 12); 2,5,6-trimethyl-2-indanmethanol (compound 14); 2,4-dimethyl-2-indanmethanol (compound 15); 2,4,6-Trimethyl-2-indanemethanol (compound 16).
These compounds were synthesized according to the procedure reported in the publication Helv. Chim. Acta 2005, 88, 3118 and in patent EP 1022265.
5-Tert-butyl-2-methyl-2-indanmethanol (compound 13):
This compound was synthesized according to the procedure reported in the publication Helv. Chim. Acta 2004, 87, 1767.

### Example 1

### Synthesis of Compounds of Formula (I)

### (−)-(R)-2,5-dimethyl-2-indanemethanol (Compound 1R) and (−)-(S)-2,5-dimethyl-2-indanemethanol (Compound 1S)

Racemic 2,5-Dimethyl-2-indanemethanol (14.4 g) was resolved in portions of 1 g on a preparative HPLC column (Chiralpack AD; 25×11 cm, 20 mm), eluting with isohexane/EtOH 95:5. After concentration to dryness, (+)-(S)-2,5-Dimethyl-2-indanemethanol (6.39 g) and (−)-(R)-2,5-Dimethyl-2-indanemethanol (6.15 g) were obtained and further purified by flash chromatography and bulb-to-bulb distillation (boiling at 1108 oven temp./0.01 mbar). The (S)- and (R)-isomers of 2,5-Dimethyl-2-indanemethanol were >99% and >98%. The absolute configuration of (+)-(S)-2,5-dimethyl-2-indanemethanol was established through X-ray diffraction, using crystals of the ester obtained from the condensation of it with (−)-camphanoyl chloride (see Helv. Chim. Acta 2005, 88, 3109).

### 2-(Methoxymethyl)-2,5-dimethyl-2,3-dihydro-1H-indene (Compound 2)

The synthesis of 2-(methoxymethyl)-2,5-dimethyl-2,3-dihydro-1H-indene was accomplished in one step starting from 2,5-dimethyl-2-indanemethanol. NaH (55% suspension in mineral oil, 0.34 g, 7.7 mmol, 1.4 eq) was washed with pentane (3 times) and suspended in THF (5.0 mL). A solution of 2,5-dimethyl-2-indanemethanol (1.0 g, 5.5 mmol, 1.0 eq) in THF (10 mL) was added dropwise and the mixture was stirred at room temperature during 0.5 hours. Mel (0.59 mL, 9.4 mmol, 1.7 eq) was added dropwise and the mixture was stirred at room temperature for 16 hours. The mixture was then diluted with Et₂O and the reaction was quenched through cautious addition of water. The organic layer was washed with sat. aqueous NaHCO₃ and brine, dried over MgSO₄, filtered and concentrated in vacuo to afford a yellow crude oil. The latter was purified by bulb-to-bulb distillation (0.30 mbar, oven temp. 75° C.) to furnish the product as a clear colorless oil (1.03 g, 5.42 mmol, 96% yield, 98% purity).

Analytical data:

¹H-NMR: 7.04 (d, J=7.56 Hz, 1H, Ar), 6.98 (s, 1H, Ar), 6.93 (d, J=7.44 Hz, 1H, Ar), 3.34 (s, 3H, OCH₃), 3.24 (s, 2H, CH₂OMe), 2.89 (d, J=9.01 Hz, 1H, ArCH₂), 2.86 (d, J=8.76 Hz, 1H, ArCH₂), 2.61 (d, J=3.66 Hz, 1H, ArCH₂), 2.58 (d, J=3.54 Hz, 1H, ArCH₂), 2.30 (s, 3H, ArCH₃), 1.16 (s, 3 H, aliphat. CH₃).

¹³C-NMR: 142.8, 139.6, 135.7, 126.9, 125.5, 124.5, 80.8, 59.3, 44.1, 43.2, 42.9, 24.7, 21.2.

### 1-(2,5-Dimethyl-2,3-dihydro1H-inden-2-yl)ethan-1-one (Compound 6)

The synthesis of 1-(2,5-dimethyl-2,3-dihydro-1H-inden-2-yl)ethan-1-one was accomplished in two steps starting from 2,5-dimethyl-2,3-dihydro-1H-indene-2-carbaldehyde. The latter was prepared according to the procedure reported in the publication Helv. Chim. Acta 2005, 88, 3118.

A solution of 2,5-dimethyl-2,3-dihydro-1H-indene-2-carbaldehyde (10.2 g, 58.8 mmol, 1.0 eq) in Et₂O (60 mL) was added dropwise to a suspension of MeMgBr (3.0 M in Et₂O, 31.4 mL, 94.0 mmol, 1.6 eq) in Et₂O (25 mL) under stirring at room temperature over a period of 1 hour. Exothermy was observed during the addition (22 to 34° C.). The resulting mixture was stirred at room temperature for 1 hour. The reaction was quenched by pouring the mixture onto ice/sat. aqueous NH₄Cl. The aqueous layer was extracted with EtOAc (3 times). The combined organic layers were washed with brine (once), dried over MgSO₄, filtered and the solvent was removed under reduced pressure to afford a pale yellow crude oil. The latter was purified by bulb-to-bulb distillation (0.14 mbar, oven temp.: 150° C.) to furnish 1-(2,5-dimethyl-2,3-dihydro-1H-inden-2-yl)ethan-1-ol as a colorless oil (10.0 g, 52.6 mmol, yield 89%; 1:1 mixture of diastereoisomers).

Analytical data:

¹H-NMR: 7.04 (dd, J=3.98, 7.51 Hz, 1H, Ar), 6.98 (d, J=4.07 Hz, 1H, Ar), 6.93 (d, J=7.63 Hz, 1H, Ar), 3.77 (q, J=6.35 Hz, 1H, CHOH), 3.01-2.85 (m, 2H, ArCH₂), 2.60 (d, J=15.74 Hz, 1H, ArCH₂), 2.47 (d, J=15.70 Hz, 1H, ArCH₂), 2.30 (s, 3H, ArCH₃), 1.61 (broad s, 1H, OH), 1.18 (d, J=6.34 Hz, 3H, CHCH₃), 1.04 (s, 3H, aliphat. CH₃).

¹³C-NMR (the signals corresponding to the two diastereoisomers are partially resolved): 142.7, 142.6, 139.5, 139.4, 135.7, 127.0, 125.6, 125.4, 124.6, 124.4, 74.4, 48.2, 43.4, 43.1, 43.0, 42.9, 21.4, 21.4, 21.2, 18.9.

1-(2,5-Dimethyl-2,3-dihydro-1H-inden-2-yl)ethan-1-ol (10.0 g, 52.6 mmol, 1.0 eq) obtained from the previous reaction was dissolved in acetone (50 mL) and the resulting clear solution was cooled to 0° C. (ice/water bath) under stirring. Jones reagent (2.7 M, 21.4 mL, 57.8 mmol, 1.1 eq) was added dropwise, at such a rate to maintain the temperature under 5° C. The reaction mixture was then stirred at room temperature for 1 additional hour. The reaction was quenched by pouring the mixture onto brine. The aqueous layer was extracted with Et₂O (3 times). The combined organic layers were washed with brine (3 times), sat. aqueous NaHCO₃ (once) and again with brine, dried over MgSO₄, filtered and concentrated in vacuo, to afford a yellow crude oil. The latter was purified by bulb-to-bulb distillation (0.12 mbar, oven temp. 120° C.) to furnish the product as a clear colorless solid (8.40 g, 43.8 mmol, 83% yield, 98% purity).

Analytical data:

¹H-NMR: 7.07 (d, J=7.60 Hz, 1H, Ar), 7.01 (s, 1H, Ar), 6.97 (d, J=7.65 Hz, 1H, Ar), 3.36 (d, J=10.45 Hz, 1H, ArCH₂), 3.33 (d, J=10.30 Hz, 1H, ArCH₂), 2.74 (d, J=4.05 Hz, 1H, ArCH₂), 2.70 (d, J=4.20 Hz, 1H, ArCH₂), 2.31 (s, 3H, COCH₃), 2.20 (s, 3H, ArCH₃), 1.30 (s, 3H, aliphat. CH₃).

¹³C-NMR: 212.0, 141.3, 138.0, 136.3, 127.4, 125.5, 124.5, 56.2, 42.6, 42.3, 25.6, 24.6, 21.2.

### (2,6-dimethyl-1,2,3,4-tetrahydro-2-naphthalenyl)methanol (Compound 7)

The synthesis of (2,6-dimethyl-1,2,3,4-tetrahydronaphthalen-2-yl)methanol was accomplished in five steps starting from p-xylene and maleic anhydride.

Maleic aldehyde (22.0 g, 224 mmol, 1.00 equivalent) was dissolved in p-xylene (279.0 mL, 2244 mmol, 10.0 eq). Di-tert-butyl peroxide (0.234 g, 1.57 mmol, 0.007 eq) was added to the resulting solution under stirring and the mixture was heated to 150° C. (bath temperature) during 5 hours. The excess p-xylene was removed by distillation under reduced pressure. The residue was purified by crystallization from a mixture of EtOAc (30 mL) and heptane (30 mL) to give the pure product as an off-white solid (21.8 g). An additional amount (8.30 g) of the pure product was obtained through bulb-to-bulb distillation of the mother liquor (0.16 mbar, over temp. 225° C.) (Total amount of the product: 73.6 g, 147 mmol, 74% yield, 99% purity).

¹H-NMR: 7.14 (d, J=7.88, 2H), 7.05 (d, J=8.00 Hz, 2H), 3.42 (m, J=2.11, 1H), 3.17 (dd, J=4.92, 14.27, 1H), 2.98 (dd, J=8.28, 14.04, 1H), 2.93 (dd, J=9.78, 18.91, 1H), 2.71 (dd, J=6.46, 18.99, 1H), 2.33 (s, 3H).

Methanesulfonic acid (100 g, 102 mmol, 11.0 eq) was added to the anhydride obtained from the previous reaction (3-(4-methylbenzyl)dihydrofuran-2,5-dione, 19 g, 93 mmol, 1.0 eq). The resulting mixture was then stirred and heated to 100° C., gradually transforming into a suspension. The mixture was cooled to 15° C. using a water bath. MeOH (70 mL) was added dropwise, while maintaining the temperature under 20° C. Once the addition was completed, the mixture was stirred at room temperature for another 15 minutes and then partitioned between brine and Et₂O. The aqueous layer was extracted with Et₂O (4 times). The combined organic layers were washed with brine (5 times), dried over MgSO₄, filtered and concentrated in vacuo to afford a red-brown crude oil. Bulb-to-bulb distillation (twice, 0.18 mbar, oven temp. 190-210° C.) furnished the pure ketoester as a pale yellow solid (13.6 g, 62.5 mmol, 67% yield).

¹H-NMR: 7.83 (s, 1H), 7.32 (dd, J=1.55, 8.05, 1H), 7.17 (d, J=7.80, 1H), 3.72 (3H, s), 3.22-3.14 (m, 3H), 2.92 (m, J=5.23, 1H), 2.80 (m, J=5.57, 1H), 2.36 (s, 3H).

The ketoester obtained from the previous reaction (methyl 6-methyl-4-oxo-1,2,3,4-tetrahydronaphthalene-2-carboxylate, 13.0 g, 59.6 mmol) was dissolved in AcOH (130 mL). Palladium on charcoal (10%, 1.2 g) was added and the resulting suspension was shaken under an atmosphere of H₂ (1 atm) during 2 days. After this time, the reaction mixture was filtered through celite, which was then washed with Et₂O. The filtrate was concentrated in vacuo to furnish a pale yellow crude oil. Bulb-to-bulb distillation (0.15 mbar, oven tem. 130° C.) afforded the pure ester (10.5 g, 51.6 mmol, 86% yield).

¹H-NMR: 6.98 (d, J=7.76, 1H), 6.92 (d, J=7.96, 1H), 6.90 (s, 1H), 3.71 (s, 3H), 2.99-2.90 (m, 2H), 2.86-2.76 (m, 2H), 2.71 (m, J=3.04, 1H), 2.28 (s, 3H), 2.19 (m, J=4.11, 1H), 1.83 (m, J=3.74, 1H).

Under a nitrogen atmosphere, the ester obtained from the previous reaction (methyl 6-methyl-1,2,3,4-tetrahydronaphthalene-2-carboxylate, 10.5 g, 51.6 mmol, 1.0 eq) was dissolved in THF (70 mL) and the resulting solution was cooled to −78° C. After 10 minutes, an LDA solution (2.0 M in THF, 33.5 mL, 67.0 mmol, 1.3 eq) was added dropwise, while maintaining the temperature under −68° C. The resulting suspension was subsequently stirred at −78° C. for 2 hours. MeI (4.2 mL, 67 mmol, 1.3 eq) was then added dropwise and the mixture was further stirred for 2 hours, while allowing the temperature to rise up to −55° C. The reaction was quenched by addition of sat. aqueous NH₄Cl. The aqueous layer was extracted with Et₂O (3 times). The combined organic layers were washed once with brine, dried over MgSO₄, filtered and concentrated in vacuo to furnish a pale yellow crude oil. Purification by column chromatography (SiO₂, elution with cyclohexane/EtOAC 24/1 to 10/1) afforded the pure product as a pale yellow solid (7.38 g, 33.4 mmol, 67% yield).

¹H-NMR: 6.97 (d, J=7.70, 1H), 6.91 (d, J=7.85, 1H), 6.89 (s, 1H), 3.65 (s, 3H), 3.19 (d, J=16.30, 1H), 2.78 (t, J=6.35, 1H), 2.61 (d, J=16.30, 1H), 2.27 (s, 3H), 2.13 (m, J=3.05, 1H), 1.76 (m, J=6.74, 1H), 1.26 (s, 3H).

LiAlH₄ (1.50 g, 39.6 mmol, 1.3 eq) was suspended in Et₂O (60 mL). The suspension was cooled under stirring to 0° C. A solution of the ester obtained from the previous reaction (methyl 2,6-dimethyl-1,2,3,4-tetrahydronaphthalene-2-carboxylate, 6.60 g, 30.2 mmol, 1.0 eq) was then added dropwise during 1 hour. The reaction mixture was stirred at room temperature for 1 additional hour and then cooled back to 0° C. Water (1.5 mL), aqueous NaOH (15% w/w, 1.5 mL), and water (4.5 mL) were carefully added in this order under vigorous stirring. The resulting suspension was further stirred at room temperature for 45 minutes. The solids were filtered off through celite and washed with Et₂O (5 times). The organic solution was concentrated in vacuo to afford a pale yellow crude oil, which was purified by bulb-to bulb distillation (0.15 mbar, oven temp. 130° C.). The pure product (4.45 g, 23.4 mmol, 77% yield, 99% purity) was obtained as a colorless oil.

¹H-NMR: 6.96-6.88 (m, 3H), 3.44 (d, J=10.68, 1H), 3.40 (d, J=10.64, 1H), 2.76 (d, J=6.44, 1H), 2.74 (d, J=6.44, 1H), 2.62 (d, J=16.33, 1H), 2.43 (d, J=16.29, 1H), 2.28 (s, 3H), 1.72-1.61 (m, 2H), 1.54 (m, =4.25, 1H), 0.97 (s, 3H).

¹³C-NMR: 135.6, 135.0, 132.3, 129.4, 129.3, 126.5, 71.3, 37.9, 34.5, 30.8, 25.8, 22.1, 20.9.

### (2,4,5-trimethyl-2,3-dihydro-1H-inden-2-yl)methanol (Compound 9)

The synthesis of (2,4,5-Trimethyl-2-indanemethanol was accomplished in six steps starting from 2,3-dimethylbenzaldehyde.

Ethyl 2-(diethoxyphosphoryl)propanoate (80.0 g, 335 mmol, 1.5 eq) was added to a stirred solution of 2,3-dimethylbenzaldehyde (30.0 g, 224 mmol, 1.0 eq) in pentane (300 mL) at room temperature. A solution of NaOEt (21% w/w in EtOH, 109 mL, 293 mmol, 1.3 eq) was subsequently added dropwise under stirring, while cooling the reaction mixture with a water bath. Once the addition was completed, the resulting mixture was stirred at reflux for 45 minutes. The reaction mixture was then cooled to 0° C. and quenched by addition of aqueous NaOH (1 N, 300 mL). The organic layer was separated and washed again with NaOH (1 N, 300 mL). The combined aqueous layers were extracted with Et₂O (3×), washed with sat. aqueous NaHCO₃, brine (twice), dried over MgSO₄, filtered and concentrated in vacuo to afford an orange crude oil. Bulb-to bulb distillation (0.15 mbar, oven temp. 150-155° C.) afforded the product as a colorless oil (46.5 g, 213 mmol, 95% yield; 94:6 mixture of E-Z isomers).

¹H-NMR (major diastereoisomer): 7.79 (s, 1H), 7.13-7.06 (m, 2H), 7.01 (m, J=3.00, m), 4.28 (q, J =7.13, 2H), 2.29 (s, 3H), 2.17 (s, 3H), 1.90 (d, J=1.40, 3H), 1.35 (t, J=7.13, 3H).

To a solution of the esters obtained in the previous reaction (ethyl 3-(2,3-dimethylphenyl)-2-methylacrylate, 46.5 g, 213 mmol) in EtOAc (50 mL) was added palladium on charcoal (10%, 1.2 g) and the resulting suspension was stirred under H₂ (40 atm) in an autoclave. The solids were then filtered off through celite and washed with CH₂Cl₂ (5 times). Removal of the solvent under reduced pressure afforded the crude product as a colorless oil (46.5 g). The latter was then dissolved in a 2.5 N NaOH solution in water/EtOH. The resulting mixture was heated to reflux. The mixture was cooled down to 0° C. with an ice/water-bath. Under stirring, concentrated aqueous HCl was added in small portions until acidic pH (pH≤1). The aqueous layer was extracted CH₂Cl₂ (4 times). The combined organic layers were washed with brine, dried over MgSO₄, filtered and concentrated in vacuo to afford an oil (38.1 g), which was not submitted to further purification. Finally, the so prepared carboxylic acid was added to polyphosphoric acid (PPA, 300 g), preheated to 100° C. The resulting mixture rapidly converted into a suspension, which was stirred at 110° C. for 1 hour. The reaction was then quenched by pouring the mixture onto ice/water, with formation of a red solution. The aqueous layer was extracted with Et₂O (4 times). The combined organic layers were washed once with brine, dried over MgSO₄, filtered and the concentrated in vacuo to afford a red-brown crude oil. Bulb-to-bulb distillation (0.15 mbar, oven temp. 140-170° C.) furnished the pure product (23.0 g, 132 mmol, 62% yield over 3 steps) as a pale yellow solid.

¹H-NMR: 7.50 (d, J=7.74, 1H), 7.17 (d, J=7.74, 1H), 3.29 (dd, J=7.74, 17.00, 1H), 2.69 (m, J=3.77, 1H), 2.58 (dd, J=1.85, 18.89, 1H), 2.35 (s, 3H), 2.23 (s, 3H), 1.30 (d, J=7.45, 3H).

The 2,4,5-trimethylindanone (22.2 g, 127 mmol, 1.0 eq) obtained from the previous reaction was dissolved in toluene (52 mL). K₂CO₃ (8.89 g, 63.7 mmol, 0.5 eq) was then added and the resulting mixture was heated to 50° C. under stirring. A solution of formaldehyde in MeOH (Formacel, 55% w/w, 10.5 mL, 204 mmol, 1.6 eq) was added dropwise and the reaction mixture was then stirred at 50° C. for 3 hours. The reaction was subsequently stopped and allowed to cool down to room temperature. The mixture was diluted with Et₂O and washed brine (3 times), dried over MgSO₄, filtered and concentrated in vacuo. Purification by column chromatography (SiO₂, elution with CH₂Cl₂) afforded the pure product (22.7 g, 111 mmol, 82%) as a colorless solid.

¹H-NMR: 7.47 (d, J=7.78, 1H), 7.16 (d, J=7.78, 1H), 3.81 (d, J=10.70, 1H), 3.61 (d, J=10.70, 1H), 3.14 (d, J=17.11, 1H), 2.77 (d, J=17.14, 1H), 2.65 (broad s, 1H), 2.36 (s, 3H), 2.24 (s, 3H), 1.23 (s, 3H).

The hydroxyketone obtained from the previous reaction (2-(hydroxymethyl)-2,4,5-trimethyl-2,3-dihydro-1H-inden-1-one, 22.3 g, 109 mmol) was dissolved in AcOH (440 mL). Palladium on charcoal (10%, 1.2 g) was added and the resulting suspension was shaken under an atmosphere of H₂ (1 atm) during 3 days. After this time, the reaction mixture was filtered through celite, which was then washed with Et₂O. The filtrate was concentrated in vacuo to furnish a pale yellow crude oil. Purification by column chromatography (SiO₂, elution with cyclohexane/EtOAC 45/5 to 40/10.) afforded the pure product (12.9 g, 67.2 mmol, 62% yield, 98% purity) as a pale yellow oil. A sample was further purified by bulb-to-bulb distillation (0.16-0.17 mbar, oven temp. 140° C.) to give a colorless oil (99% purity)

¹H-NMR: 6.94 (d, J=7.60, 1H), 6.90 (d, J=7.55, 1H), 3.51 (s, 2H), 2.89 (d, J=14.07, 1H), 2.86 (d, J=13.65, 1H), 2.65 (d, J=15.85, 1H), 2.60 (d, J=16.10, 1H), 2.24 (s, 3H), 2.14 (s, 3H), 1.65 (s, 1H), 1.18 (s, 3H).

¹³C-NMR: 141.5, 139.7, 134.1, 132.6, 128.0, 121.7, 70.9, 44.4, 42.9, 42.0, 24.4, 19.6, 15.8.

### (2,5-dimethyl-2-indanyl)methyl acetate (Compound 10)

The synthesis of (2,5-dimethyl-2,3-dihydro-1H-inden-2-yl)methyl acetate was accomplished in one step starting from 2,5-dimethyl-2-indanemethanol.

A solution of 2,5-dimethyl-2-indanemethanol (0.57 g, 3.1 mmol) in pyridine (5 mL) and acetic anhydride (5 mL) was stirred at room temperature for 3 hours. The mixture was then concentrated under reduced pressure and the residue was evaporated three times from toluene to obtain a crude oil. The latter was purified through bulb-to-buld distillation (0.35 mbar, oven temp. 100-135° C.) to furnish the product (0.63 g, 2.5 mmol, 87% yield, 94% purity) as an oil.

¹H-NMR: 7.04 (d, J=7.56, 1H) 6.98 (s, 1H), 6.94 (d, J=7.60, 1H), 3.99 (s, 2H), 2.89 (dd, J=4.16, 15.79, 2H), 2.63 (d, J=15.63, 2H), 2.30 (s, 3H), 2.05 (s, 3H), 1.16 (s, 3H).

¹³C-NMR: 171.2, 142.2, 139.0, 135.9, 127.1, 125.5, 124.5, 71.3, 43.3, 43.0, 42.7, 24.3, 21.2, 20.9.

### 2-ethyl-5-methyl-2-indanmethanol (Compound 11)

The synthesis of (2-ethyl-5-methyl-2,3-dihydro-1H-inden-2-yl)methanol was accomplished in four steps starting from 5-methylindanone.

NaH (55% dispersion in mineral oil, 3.9 g, 90 mmol, 2.2 eq) was washed with pentane (3 times) and suspended in a mixture of toluene (50 ml) and 1,2-dimethoxyethane (20 ml). Dimethyl carbonate (9.0 g, 100 mmol) was added and the mixture was heated to 60° C. A solution of 5-methylindanone (6.0 g, 41 mmol) in toluene (20 ml) was added dropwise over a period of 1 hour, while maintaining the temperature between 60-80° C. (H₂ evolution). After stirring for 2 hours at 80° C., the mixture was cooled, diluted with ether and saturated aqueous NaHCO₃. The organic layer was washed with brine (twice), dried over Na₂SO₄ and concentrated under reduced pressure to afford an oil. Bulb-to-bulb distillation (0.2 mbar, oven temp. 175° C.) provided methyl 5-methyl-1-oxo-2,3-dihydro-1H-indene-2-carboxylate as an oil (4.92 g, 24.1 mmol, 59% yield). The product was crystallized from ether-pentane at −30° C. to afford colorless crystals (mp 42-46° C.).

¹H-NMR: 7.65 (d, J=7.89, 1H), 7.29 (s, 1H), 7.20 (d, J=7.92, 1H), 3.78 (s, 3H), 3.72 (dd, J=4.02, 8.23, 1H), 3.50 (dd, J=3.96, 17.25, 1H), 3.31 (dd, J=8.25, 17.25, 1H), 2.44 (s, 3H).

¹³C-NMR: 199.0, 169.7, 154.1, 146.9, 133.0, 129.1, 126.9, 124.5, 53.3, 52.7, 30.1, 22.1.

To a stirred solution of methyl 5-methyl-1-oxo-2,3-dihydro-1H-indene-2-carboxylate (2.0 g, 10 mmol, 1.0 eq) in THF at room temperature was added K₂CO₃ (2.8 g, 20 mmol, 2.0 eq) and ethyl iodide (2.34 g, 15 mmol, 1.5 eq) and the mixture was heated to reflux (65° C.) during 20 hours. The mixture was then cooled to room temperature, diluted with ether, and washed with saturated aqueous NaHCO₃ and brine. The organic layer was dried over Na₂SO₄ and concentrated under reduced pressure to afford a yellow crude oil. Bulb-to-bulb distillation (0.2 mbar, oven temp. 150° C.) afforded methyl 2-ethyl-5-methyl-1-oxo-2,3-dihydro-1H-indene-2-carboxylate as an oil (2.20 g, 9.48 mmol, 93% yield, 98% purity). Crystallization from ether at −30° C. gave colorless crystals (mp 67-68° C.).

¹H-NMR: 7.65 (d, J=7.85, 1H), 7.29 (s, 1H), 7.20 (d, J=7.81, 1H), 3.68 (s, 3H), 3.66 (d, J=19.52, 1H), 3.03 (d, J=17.39, 1H), 2.45 (s, 3H), 2.14 (m, J=7.18, 1H), 1.93 (m, J=7.21, 1H), 0.87 (t, J=7.44, 3H).

¹³C-NMR: 202.1, 171.8, 153.6, 146.7, 133.2, 129.0, 126.7, 124.5, 61.2, 52.6, 36.1, 27.9, 22.1, 9.0.

To a solution of methyl 2-ethyl-5-methyl-1-oxo-2,3-dihydro-1H-indene-2-carboxylate (1.6 g, 7.1 mmol) in acetic acid (20 ml) was added 10% Pd—C (0.2 g) and the mixture was stirred under an atmosphere of H₂ (1 atm) at room temperature over a period of 110 hours. The catalyst was filtered off and the filtrate was concentrated under reduced pressure to afford a yellow crude oil. Bulb-to-bulb distillation (0.2 mbar, oven temp. 125° C.) gave methyl 2-ethyl-5-methyl-2,3-dihydro-1H-indene-2-carboxylate as a colorless oil (1.40 g, 6.41 mmol, 87% yield, 97% purity).

¹H-NMR: 7.04 (d, J=7.60, 1H), 6.98 (s, 1H), 6.94 (d, J=7.71), 3.68 (s, 3H), 3.41 (dd, J=4.72, 16.13, 2H), 2.84 (d, J=16.03, 2H), 2.30 (s, 3H), 1.76 (q, J=7.43, 2H), 0.86 (t, J=7.44, 3H).

¹³C-NMR: 177.4, 141.5, 138.3, 136.0, 127.2, 125.1, 124.1, 55.0, 51.9, 41.6, 41.4, 31.6, 21.2, 9.9.

To a stirred suspension of LiAlH₄ (220 mg, 5.8 mmol, 1.0 eq) in ether (20 ml) at room temperature was added dropwise a solution of methyl 2-ethyl-5-methyl-2,3-dihydro-1H-indene-2-carboxylate (1.30 g, 5.8 mmol, 1.0 eq) in ether (10 ml) and the mixture was stirred at room temperature during 0.5 hours. The mixture was diluted with ether, acetone (0.5 ml) was added followed by 1.0 N aqueous NaOH (1.1 ml) and the mixture was stirred at room temperature during 0.5 hours. Na₂SO₄ was added, the solids were filtered off and the filtrate concentrated under reduced pressure to provide an oil. Bulb-to-bulb distillation (0.2 mbar, oven temp. 140° C.) gave (2-ethyl-5-methyl-2,3-dihydro-1H-inden-2-yl)methanol as a colorless oil (1.10 g, 5.78 mmol, 98% yield, >98% purity).

¹H-NMR: 7.03 (d, J=7.60, 1H), 6.97 (s, 1H), 6.92 (d, J=7.60, 1H), 3.49 (d, J=3.96, 2H), 2.76 (dd, J=4.09, 16.12, 2H), 2.68 (dd, J=2.18, 16.22, 2H), 2.29 (s, 3H), 1.68 (br s, 1H), 1.59 (q, J=7.48, 2H), 0.88 (t, J=7.49, 3H).

¹³C-NMR: 142.7, 139.4, 135.7, 127.0, 125.4, 124.4, 67.7, 48.1, 40.6, 40.3, 29.0, 21.2, 9.0.

### (5-methoxy-2-methyl-2,3-dihydro-1H-inden-2-yl)methanol (Compound 17)

The synthesis of (5-methoxy-2-methyl-2,3-dihydro-1H-inden-2-yl)methanol was accomplished in four steps starting from 3-(4-methoxyphenyl)-2-methylpropanal.

Sodium acetate (92.0 g, 1.12 mol, 0.8 eq) was added to a solution of 3-(4-methoxyphenyl)-2-methylpropanal (250 g, 1.4 mol, 1.0 eq) in toluene (575 mL). The resulting mixture was heated to 30° C. and peracetic acid (117 g, 1.54 mol, 1.1 eq) was added dropwise under stirring over a period of 3 hours. The mixture was then stirred at 30° C. for one hour. The mixture was then washed with water (twice), 5% w/w aqueous Na₂SO₃ (twice), and water. The resulting pale yellow crude oil was submitted to bulb-to-buld distillation (0.1 mbar, oven temp. 130-145° C.) to afford 3-(4-methoxyphenyl)-2-methylpropanoic acid (244 g, 1.29 mol, 89% yield, 99% purity) as an oil.

¹H-NMR: 11.66 (br s, 1H), 7.09 (d, J=8.60, 2H), 6.82 (d, J=8.64, 2H), 3.76 (s, 3H), 3.00 (dd, J=6.38, 13.46, 1H), 2.71 (m, J=7.06, 1H), 2.61 (dd, J=7.92, 13.44, 1H), 1.16 (d, J=6.96, 3H).

¹³C-NMR: 182.8, 158.2, 131.0, 129.9, 113.8, 55.2, 41.5, 38.4, 16.4.

3-(4-Methoxyphenyl)-2-methylpropanoic acid (170 g, 875 mmol) was added dropwise to polyphosphoric acid (150 g) under stiffing at 95° C. over a period of 55 minutes. The resulting red mixture was then cooled to room temperature and water (140 mL) was added. Toluene (140 mL) was added and the biphasic mixture was stirred before removing the aqueous layer. The organic layer was washed with water and saturated aqueous NaHCO₃. The resulting mixture was concentrated under reduced pressure, diluted in MTBE and the organic solution was washed with 10% w/w aqueous NaOH and water (4 times). Upon removal of the volatiles under reduced pressure, the resulting crude oil was submitted to bulb-to-bulb distillation (0.1 mbar, oven temp. 90-120° C.) to afford 6-methoxy-2-methyl-2,3-dihydro-1H-inden-1-one (67.3 g, 375 mmol, 43% yield, 98% purity) as an oil.

¹H-NMR: 7.32 (m, 1H), 7.18-7.15 (m, 2H), 3.82 (s, 3H), 3.31 (dd, J=7.60, 16.65, 1H), 2.72 (m, J=4.34, 1H), 2.64 (dd, J=3.72, 16.65, 1H), 1.30 (d, J=7.48, 3H).

¹³C-NMR: 209.4, 159.4, 146.2, 137.4, 127.2, 124.0, 105.1, 55.5, 42.8, 34.3, 16.3.

6-Methoxy-2-methyl-2,3-dihydro-1H-inden-1-one (74.5 g, 383 mmol, 1.0 eq) was dissolved in toluene (170 mL) and K₂CO₃ (26.5 g, 190 mmol, 0.5 eq) was added to the resulting solution. The latter was heated to 60° C. and formaldehyde (55% w/w solution in MeOH, 20.9 g, 380 mmol, 1.0 eq) was then added dropwise over a period of 90 minutes. The mixture was stirred at the same temperature for additional 60 minutes and it was then allowed to cool down to room temperature. The organic mixture was washed with water, 1% w/w aqueous H₂SO₄ (twice), water (3 times) and concentrated under reduced pressure. Upon crystallization from toluene, 2-(hydroxymethyl)-6-methoxy-2-methyl-2,3-dihydro-1H-inden-1-one (75.6 g, 206 mmol, 96% yield, >99% purity) was obtained.

¹H-NMR: 7.33 (d, J=8.44, 1H), 7.18 (dd, J=2.56, 8.32, 1H), 7.10 (d, J=2.52, 1H), 3.83 (dd, J=6.74, 10.70, 1H), 3.78 (s, 3H), 3.59 (dd, J=5.08, 10.72, 1H), 3.20 (d, J=16.85, 1H), 2.88 (dd, J=5.26, 6.58, 1H), 2.79 (d, J=16.81, 1H), 1.21 (s, 3H).

¹³C-NMR: 211.1, 159.4, 146.3, 136.9, 127.3, 124.6, 105.2, 67.8, 55.5, 51.9, 37.2, 20.7.

To a solution of 2-(hydroxymethyl)-6-methoxy-2-methyl-2,3-dihydro-1H-inden-1-one (17 g, 81 mmol) in EtOH (95 ml) was added 5% Pd—C (1.66 g) and the mixture was stirred under an atmosphere of H₂ (1 atm) at 60° C. over a period of 70 hours. The catalyst was filtered off and the filtrate was concentrated under reduced pressure. The resulting crude product was recrystallized from petroleum ether (60-80)/toluene (3/1) to provide (5-methoxy-2-methyl-2,3-dihydro-1H-inden-2-yl)methanol (7.3 g, 37 mmol, 46% yield).

¹H-NMR: 7.04 (d, J=8.12, 1H), 6.71 (m, 1H), 6.67 (dd, J=2.40, 8.16, 1H), 3.75 (s, 3H), 3.48 (s, 2H), 2.84 (dd, J=15.79, 20.67, 2H), 2.58 (dd, J=15.38, 15.3, 2H), 2.08 (s, 1H), 1.15 (s, 3H).

¹³C-NMR (100 MHz, CDCl₃) δ 158.6, 144.0, 134.5, 125.2, 112.1, 110.3, 70.5, 55.3, 45.4, 42.9, 41.9, 24.0.

### Example 2

Nose Receptor Based Screening Methods and Results

Identification of malodor-sensitive olfactory neurons was performed as previously described in WO 2014/210582. Identification of antagonist compounds that inhibit the response of malodor-sensitive olfactory neurons to the target malodor was performed according to Kajiya et al (2001) (K. Kajiya, el al in The Journal of Neuroscience, (2001) 21, 6018-6025).

### Experiment 1: Identification of Indole Olfactory Receptor Antagonists Compound 1, Compound 2, and Compound 3

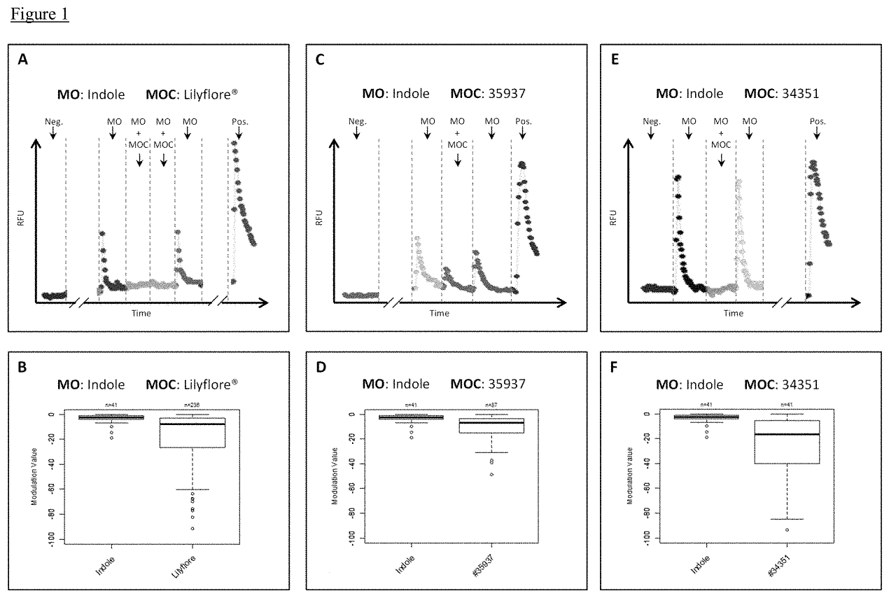

Ca²⁺ imaging traces of individual olfactory sensory neurons and their inhibition to Compound 1, Compound 2 or Compound 3 (MOC) are shown in FIG. 1 plots (A, C, E). Inhibition of the population of indole -response olfactory sensory neurons is shown in FIG. 1 plots (B, D, F).

Olfactory sensory neurons were stimulated with 25 μM indole (MO) and 125 μM MOC either alone or as a binary mixture. By comparing the peak value of the calcium-induced fluorescence ratio change induced by exposure to the target MO compound to that of the mixture of MO+MOC candidate, a “modulation value” was calculated. The larger the difference between the two peak values, the greater the magnitude of the modulation value. If the peak value for the MO was larger than that of the MO+MOC, the modulation value was negative, whilst the inverse produced positive modulation values. A modulation value was calculated for each cell responding to the positive-control stimulus forskolin (Pos) and the MO compound, but not to the negative-control buffer stimulus (Neg). For each candidate MOC compound, a baseline ‘modulation value’ was obtained by repeated stimulations of olfactory neurons with indole alone (left box plot in B, D, F). The percentage of target malodor-responsive cells with negative modulation values less than −10% was plotted on a bar chart. Population data are represented as box plots, where the interquartile range (25-75^{th} percentiles) of olfactory sensory neuron modulation is contained within the box, with the median indicated by the black bar and the 95^{th} percentile by the arm.

### Experiment 2: Identification of Antagonists of Feces Malodors Compounds Using Olfactory Receptors

TABLE 1
Summary of the results obtained on the screening of antagonist
compounds for indole, skatole and dimethyl trisulfide (DMTS):

| | Proportion³⁾ of | Proportion³⁾ of | Proportion³⁾ of |
|---|---|---|---|
| MOC Compound | Indole OSNs²⁾ | Skatole OSNs²⁾ | DTMS OSNs²⁾ |
| 17 | 23% | | |
| 16 | 14% | | |
| 15 | 17% | | |
| 14 | 20% | | |
| 13 | 54% | | |
| 12 | 57% | | |
| 11 | 35% | | |
| 10 | 42% | | |
| 9 | 78% | | |
| 8 | 30% | | |
| 7 | 54% | | |
| 6 | 69% | 92% | 94% |
| 5 | 77% | 87% | 82% |
| 4 | 41% | | |
| 3 | 41% | | |
| 2 | 43% | | |
| 1 | 56% | 82% | 69% |

¹⁾missing data mean the compound was not tested against the target malodor
²⁾OSN means Olfactory Sensory Neurons
³⁾the percentage of the Malodor-responsive olfactory neuron population that was inhibited by more than 10% (i.e. with modulation values less than −10%) was plotted.

Olfactory sensory neurons were stimulated with 25 μM indole malodor and 125 μM candidate MOC compound as a binary mixture.

Olfactory sensory neurons were stimulated with 50 μM skatole malodor and 250 μM candidate MOC compound as a binary mixture.

Olfactory sensory neurons were stimulated with 50 μM DMTS malodor and 250 μM candidate MOC compound as a binary mixture.

### Example 3

### Olfactometry Based Screening Methods and Results

Air dilution olfactometry was used to measure all psychophysical data of individual and mixed odorants. Odorized flows of air with precisely set concentrations were prepared by the evaporation of a known flux of odorant in a determined flow of air. The flux of odorant was delivered through a microsyringe operated by a calibrated micromotor into a heated vessel under a steady nitrogen flow. The odorant was vaporized and swept away by the nitrogen, and this primary flow was later diluted with humidified air to the desired concentration. Odorants can be presented one by one in olfactometers (see as a reference “Multidimensional visualization of physical and perceptual data leading to a creative approach in fragrance development”, C. Vuilleumier, M. van de Waal, H. Fontannaz, I. Cayeux and P. A. Rebetez, in Perfumer & Flavourist, 33, 55 (2008)); alternatively, a machine blending up to 12 flows of odorants in variable and adjustable proportions could be used. A sniffing outlet delivered a continuous and adjustable odorized air flow. The upper working limit was determined by the vapor pressure of the odorants at room temperature. The odorized flow was delivered at a temperature of 26° C., close to the temperature within the nose. The combination of air (540 l/h) and nitrogen (60 l/h) represented a total gas flow of 600 l/h with a relative humidity of 50%. The speed of injection of the solutions in the evaporation chamber was modulated and controlled for each subject and adjusted to obtain a medium perceived intensity (see above reference, for instance FIG. 4).

Standardized psychophysical procedures were used to determine olfactory detection thresholds (triangle testing) or perceived intensity, after a training period (see above reference).

A method was designed as an iterative process to obtain dose-response relationship and odor detection threshold of perfumery ingredients or malodorants with a minimum number of experiments (see FIG. 5 in the above reference).

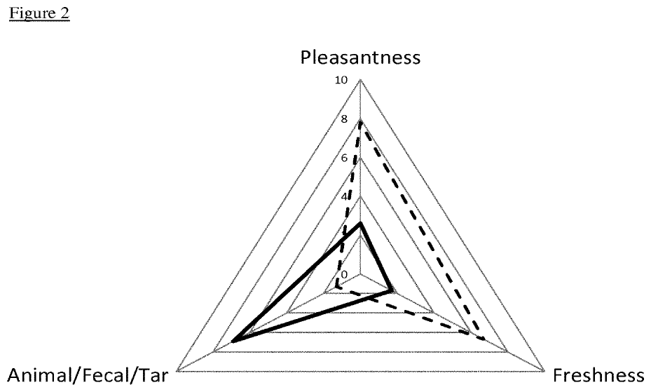

FIG. 2 reports the radar plot of indole alone and indole+compound 1 obtained with a panel of 13 persons using a sensory protocol to evaluate the potential antagonists versus indole. The protocol involves two steps:
Step 1: the participants evaluated the tested ingredients and indole independently to adjust their individualized concentrations by changing the rate of injection (see above description and reference) to elicit a medium intensity perception for each chemical. These concentrations, corresponding to iso-intense levels, were applied in the second step of the process. The range of concentrations to be submitted to subjects was determined from dose-response relationships for the selected ingredients and indole. In the present experiment the molar ratio Compound 1/indole varied according to the individuals from about 1/1 to 10/1 (typical preferred individual range is between 2.5/1 and 4.5/1).
Step 2: Blind sensory evaluations were set; no information was disclosed to the participants on the submitted odorous stimuli. The subjects had to evaluate first indole alone at its individualized concentration and rate the following three descriptors on a linear labeled scale:
Pleasantness (From «Very unpleasant» to «Very pleasant»)
Freshness (From «No Freshness» to «Very fresh»)
Malodor character: Animal/Fecal/Tar (From «No . . . » to «Very . . . »)

The next submission, 30 seconds after the previous one to avoid odor adaptation, involved the simultaneous injection of indole and of the tested ingredient at individualized concentrations. The same descriptors were rated.

By applying the same method to various compounds were obtained the results reported in Table 2 herein below.

TABLE 2
Sensory results of Indole vs (Indole + invention's Compound)

| | Animal/Fecal/Tar | | Freshness | Pleasantness | |
|---|---|---|---|---|---|
| Compound | score Reduction ²⁾ | | score | score | Compound/Indole ¹⁾ |
| Indole | 6.9 ± 0.2 | 0 | 2.2 ± 0.2 | 2.6 ± 0.2 | — |
| alone | | | | | |
| 2 | 3.5 ± 1.5 | 50 | 4.7 ± 1.3 | 3.9 ± 0.8 | 23.9 |
| 3 | 3.1 ± 1.3 | 55 | 5.2 ± 1.5 | 6.0 ± 1.4 | 26.5 |
| 5 | 2.7 ± 1.0 | 61 | 5.2 ± 1.2 | 5.7 ± 1.1 | 51.1 |
| 16 | 2.6 ± 1.4 | 62 | 6.2 ± 1.6 | 6.4 ± 1.3 | 4.9 |
| 6 | 2.2 ± 1.2 | 68 | 5.2 ± 1.6 | 5.8 ± 1.4 | 10.7 |
| 4 | 2.1 ± 1.1 | 70 | 6.6 ± 1.5 | 7.2 ± 1.2 | 12.1 |
| 1 | 1.3 ± 0.5 | 81 | 6.7 ± 0.9 | 7.8 ± 0.7 | 3.0 |

¹⁾ Median of individual molar concentrations ratios (Compound/Indole)
²⁾ In percentage

The best performers can be defined as being the one providing the highest reduction of the Animal/Fecal/Tar character when tested at the iso-intense levels.

Alternatively the best performer can be defined as being the one providing the lowest molar ratio vs. indole when tested at the iso-intense levels.

1. A method to modify, suppress, reduce, decrease or mask a toilet malodor comprising the step of releasing into the air or over a surface, or to the malodor source, an effective amount of at least a compound of formula 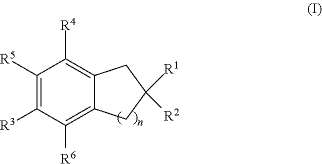
wherein n represents 1 or 2;
R¹ represents a hydrogen atom or a methyl or ethyl group;
R² represents a CH₂OR⁷ or a R⁸CO group, R⁷ being a hydrogen atom or a C₁₋₃ hydrocarbon group or a R⁸CO group, and R⁸ being a hydrogen atom or a C₁₋₃ hydrocarbon group;
R³ represents a hydrogen atom or a C₁₋₄ hydrocarbon group or a C₁₋₃ alkoxyl group; and
each of R⁴, R⁵ and R⁶ represents, independently from each other, a hydrogen atom or a C₁₋₃ alkyl group.

2. The method according to claim 1, wherein the compound (I) is a compound of formula 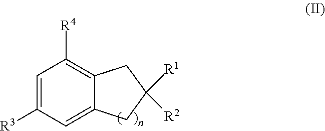
wherein n represents 1 or 2;
R¹ represents a hydrogen atom or a methyl group;
R² represents a CH₂OR⁷ or a R⁸CO group, R⁷ being a hydrogen atom or a methyl or ethyl group or a R⁸CO group, and R⁸ being a methyl or ethyl group;
R³ represents a hydrogen atom or a C₁₋₄ alkyl group; and
R⁴ represents a hydrogen atom or a methyl group.

3. The method according to claim 1, wherein the compound (I) is a compound of formula 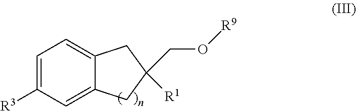
wherein n represents 1 or 2;
R¹ represents a hydrogen atom or a methyl group;
R⁹ represents a hydrogen atom or a methyl or ethyl group or a CH₃CO group; and
R³ represents a hydrogen atom or a methyl or ethyl group.

4. The method according to claim 1, wherein the compound is a C₁₁₋₁₃ compound.

5. The method according to claim 1, wherein the compound is 2,5-dimethyl-2-indanemethanol, (2,5-dimethyl-2,3-dihydro-1h-inden-2-yl)methyl methyl ether, (2-methyl-2,3-dihydro-1h-inden-2-yl)methanol, (5-methyl-2,3-dihydro-1h-inden-2-yl)methanol, (2-methyl-2,3-dihydro-1h-inden-2-yl)methyl acetate, 1-(2,5-dimethyl-2,3-dihydro-1h-inden-2-yl)ethanone, (2,4,6-trimethyl-2,3-dihydro-1H-inden-2-yl)methanol and/or (2,6-dimethyl-1,2,3,4-tetrahydro-2-naphthalenyl)methanol.

6. The method according to claim 1, wherein the toilet malodor is generated by the presence of skatole, C₁₋₇ aliphatic carboxylic acids, methyl morpholines, thioglycolic acid, cresols, C₁₋₄ dialkyl sulfide or disulfide or trisulfide, indole, and/or C₁₋₇ thiols, or mixtures thereof.

7. The method according to claim 1, wherein the toilet malodor is generated by the presence of skatole, p-cresol, dimethyl sulfide or disulfide or trisulfide, indole, or mixtures thereof.

8. A compound of formula 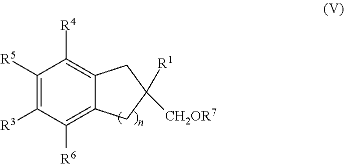
wherein n represents 1 or 2;
R¹ represents a hydrogen atom or a methyl or ethyl group;
R⁷ being a C₁₋₃ hydrocarbon group;
R³ represents a hydrogen atom or a C₁₋₄ hydrocarbon group or a C₁₋₃ alkoxyl group; and
each of R⁴, R⁵ and R⁶ represents, independently from each other, a hydrogen atom or a C₁₋₃ alkyl group;
provided that 2-methoxy-2,3-dihydro-1H-indene is excluded.

9. A MOC composition comprising:
i) as a MOC ingredient, at least one compound of formula (I), according to claim 1;
ii) at least one ingredient selected from the group consisting of a perfumery carrier and a perfumery base; and
iii) optionally at least another MOC compound; and
iv) optionally at least one perfumery adjuvant.

10. A MOC consumer product comprising, as an active ingredient, at least one compound of formula (I), according to claim 1.

11. The MOC consumer product according to claim 10, wherein the MOC consumer product is selected amongst a fabric care product, a toilet paper or napkin, an air freshening product, a surface care product and/or a pet-litter.

12. The MOC consumer product according to claim 10, wherein the MOC consumer product is selected amongst:
a fabric care product in the form of a liquid detergent, a powder detergent, detergent tablets, a detergent bar, a detergent paste, a liquid fabric softener, fabric softener sheets, a fabric scent booster, a laundry pre-treatment, a fabric refresher, an ironing water, a laundry bleach, a carpet powder or a carpet cleaner;
an air freshening product in the form of an air freshener spray, a gel air freshener, a liquid-wick air freshener, a solid air freshener comprising a porous substrate, a liquid or gel air freshener comprising a permeable membrane, an electrically operated air freshener, and a dual purpose air freshener/disinfectant spray; and/or
a surface care product in the form of an all-purpose cleaner, a furniture polish, a wood floor cleaner, a toilet care product.
